(19) United States
(12) Patent Application Publication
(10) Pub. No.: US 2010/0262234 A1
(43) Pub. Date: Oct. 14, 2010

(54) Posterior chamber phakic intraocular lens

(76) Inventors: Son Trung Tran, Arlington, TX (US); Yin Yang, Arlington, TX (US)

Correspondence Address: ALCON, IP LEGAL, TB4-8, 6201 SOUTH FREEWAY, FORT WORTH, TX, 76134, US

(21) Appl. No.: 12/566,183

(22) Filed: Sep. 24, 2009

Related U.S. Application Data
(63) Continuation of application No. 11/334,308, filed on Jan. 18, 2006, now abandoned.

(51) Int. Cl. A61F 2/16

(52) U.S. Cl. 623/6.38

(57) ABSTRACT

A posterior chamber phakic lens made from an elastomeric, foldable, highly biocompatible and permeable material. The lens has a generally circular optic and a plurality of integrally formed, filament-like haptics. The haptics project posteriorly from the optic and contain a raised feature or ridge that is sandwiched between the posterior iris and the zonules when implanted in an eye. Such a construction has a low vaulting force under compression, is size insensitive, provides for a stable lens once implanted in the eye, helps to avoid pupillary blockage and allows for improved aqueous flow around the natural lens.

This application is a continuation of U.S. Application Ser. No. 11/334,308 filed Jan. 18, 2006.

### BACKGROUND OF THE INVENTION

This invention relates generally to the field of intraocular lenses (IOL) and, more particularly, to posterior chamber phakic IOLs.

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

The optical power of the eye is determined by the optical power of the cornea and the crystalline lens. In the normal, healthy eye, sharp images are formed on the retina (emmetropia). In many eyes, images are either formed in front of the retina because the eye is abnormally long (axial myopia), or formed in back of the retina because the eye is abnormally short (axial hyperopia). The cornea also may be asymmetric or toric, resulting in an uncompensated cylindrical refractive error referred to as corneal astigmatism. In addition, due to age-related reduction in lens accommodation, the eye may become presbyopic resulting in the need for a bifocal or multifocal correction device.

In the past, axial myopia, axial hyperopia and corneal astigmatism generally have been corrected by spectacles or contact lenses, but there are several refractive surgical procedures that have been investigated and used since 1949. Barraquer investigated a procedure called keratomileusis that reshaped the cornea using a microkeratome and a cryolathe. This procedure was never widely accepted by surgeons. Another procedure that has gained widespread acceptance is radial and/or transverse incisional keratotomy (RK or AK, respectively). Recently, the use of photoablative lasers to reshape the surface of the cornea (photorefractive keratectomy or PRK) or for mid-stromal photoablation (Laser-Assisted In Situ Keratomileusis or LASIK) have been approved by regulatory authorities in the U.S. and other countries. All of these refractive surgical procedures cause an irreversible modification to the shape of the cornea in order to effect refractive changes, and if the correct refraction is not achieved by the first procedure, a second procedure or enhancement must be performed. Additionally, the long-term stability of the correction is somewhat variable because of the variability of the biological wound healing response between patients.

Several companies are investigating implantable posterior chamber phakic IOLs, including the Staar ICL lens and the Medennium PRL lens. These and other posterior chamber phakic lenses are described in U.S. Pat. No. 4,769,035 (Kelman), U.S. Pat. No. 6,015,435 (Valunin, et al.) and U.S. Pat. No. 6,106,553 (Feingold), the entire contents of which being incorporated herein by reference. The clinic experience with commercially available posterior chamber phakic lenses has not been entirely satisfactory due to pupillary block, pigment deposition, the need to accurately size the lens, unwanted rotation of the lens and the development of traumatic cataract.

Therefore, a need continues to exist for a safe, stable and biocompatible posterior chamber phakic intraocular lens.

### BRIEF SUMMARY OF THE INVENTION

The present invention improves upon the prior art by providing a posterior chamber phakic lens made from an elastomeric, foldable, highly biocompatible and permeable material. The lens has a generally circular optic and a plurality of integrally formed, filament-like haptics. The haptics project posteriorly from the optic and contain a raised feature or ridge that is sandwiched between the posterior iris and the zonules when implanted in an eye. Such a construction has a low vaulting force under compression, is size insensitive, provides for a stable lens once implanted in the eye, helps to avoid pupillary blockage and allows for improved aqueous flow around the natural lens.

Accordingly, one objective of the present invention is to provide a safe and biocompatible intraocular lens.

Another objective of the present invention is to provide a safe and biocompatible intraocular lens that is easily implanted in the posterior chamber.

Still another objective of the present invention is to provide a safe and biocompatible intraocular lens that is stable in the posterior chamber.

Still another objective of the present invention is to provide a safe and biocompatible intraocular lens that does not need highly accurate sizing.

These and other advantages and objectives of the present invention will become apparent from the detailed description and claims that follow.

### BRIEF DESCRIPTION OF THE DRAWINGS

FIG. 1 is an enlarged top plan view of the lens of the present invention.

FIG. 2 is an enlarged side view of the lens of the present invention.

FIG. 3 is an enlarged cross-sectional view of the lens of the present invention taken at line 3-3 in FIG. 1.

FIG. 4 is an enlarged cross-sectional view of a first alternative haptic design for the lens of the present invention.

FIG. 5 is an enlarged cross-sectional view of a second alternative haptic design for the lens of the present invention.

### DETAILED DESCRIPTION OF THE PREFERRED EMBODIMENTS

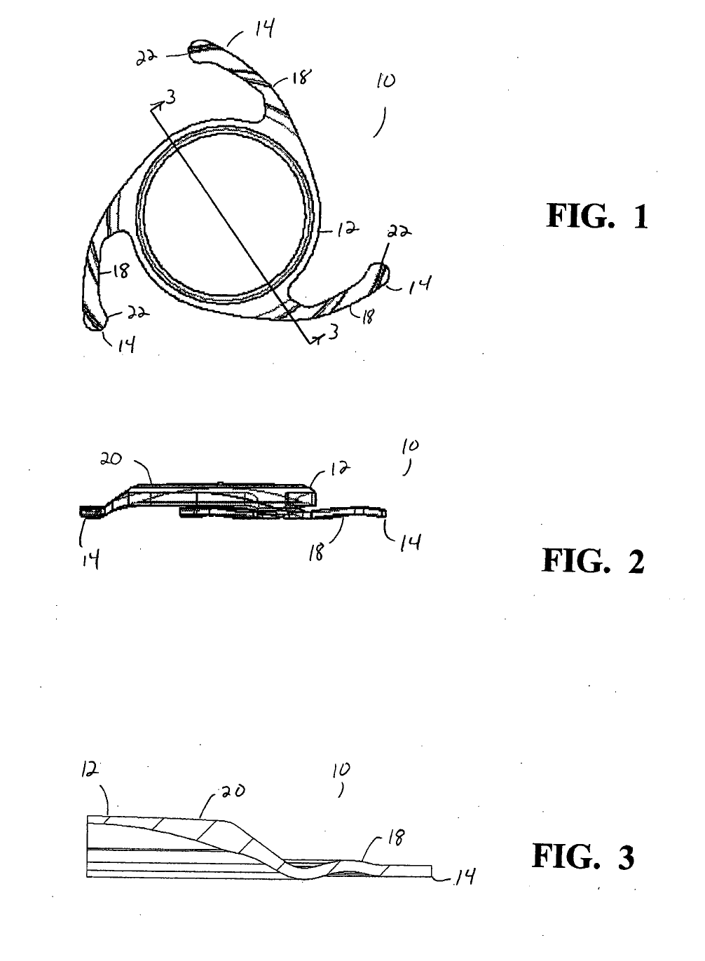

As best seen in FIGS. 1, 2 and 3, lens 10 of the present invention generally includes optic 12 and a plurality of filament-like haptics 14 integrally formed with optic 12. Optic 12 may be of any suitable size, such as between 4.5 mm and 6.5 mm in diameter, and may be biconcave, biconvex, concave/convex or any other suitable geometry. Optic 12 may also contain refractive or diffractive features, such features being well-known in the art. Lens 10 is preferably formed in any suitable overall length, for example, around 12 millimeters, for implantation in the posterior chamber in front of the natural lens, from a soft, foldable material such as a hydrogel, silicone or soft acrylic, such diameters and materials being well-known in the art. As best seen in FIGS. 2 and 3, haptics 14 project or vault posteriorly from optic 12, so as to locate optic 12 anteriorly of haptics 14 once implanted in an eye.

As best seen in FIG. 3, haptics 14 are not planar and contain raised portion or ridge 18. Ridge 18 projects anteriorly from distal tips 22 of haptics 14 but remains posterior of anterior face 20 of optic 12. Ridge 18, because of the flexible nature of the material used to manufacture haptic 14, is soft and spring-like, offering little resistance to compression by the iris. Also, ridge 18 acts like a hinge to reduce vaulting of optic 14 upon compression of haptics 14. Such a construction is size insensitive, provides for a stable lens once implanted in the eye, helps to avoid pupillary blockage and allows for improved aqueous flow around the natural lens.

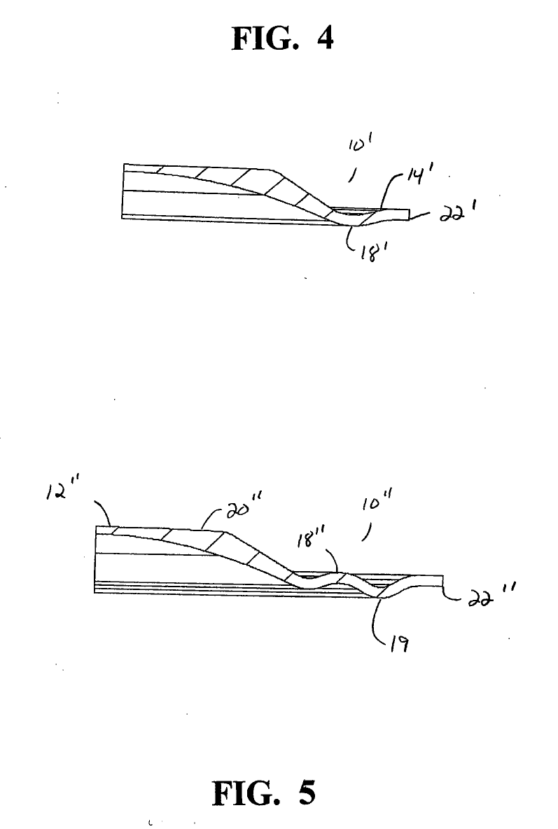

Alternatively, as seen in FIG. 4, haptics 14′ of lens 10′ contains raised portion or ridge 18′. Ridge 18′ projects posteriorly from distal tips 22′ of haptics 14′. In yet another embodiment, best seen in FIG. 5, lens 10″ contains haptics 14″ having first ridge 18″ that projects anteriorly from distal tips 22″ of haptics 14″ but remains posterior of anterior face 20″ of optic 12″ and second ridge 19 that projects posteriorly from distal tips 22″ of haptics 14″.

This description is given for purposes of illustration and explanation. It will be apparent to those skilled in the relevant art that changes and modifications may be made to the invention described above without departing from its scope or spirit.

We claim:

1. An intraocular lens, comprising;
a) an optic having an anterior face;
b) a plurality of haptics having distal tips, the haptics integrally formed with the optic and projecting outwardly and posteriorly from the optic; and
c) a ridge formed the haptics between the optic and the distal tips, the ridge projecting anteriorly of the tips, but posteriorly of the anterior face of the optic.

2. The lens of claim 1 wherein the lens is formed from a soft, foldable material.

3. An intraocular lens, comprising;
a) an optic having an anterior face;
b) a plurality of haptics having distal tips, the haptics integrally formed with the optic and projecting outwardly and posteriorly from the optic; and
c) a ridge formed the haptics between the optic and the distal tips, the ridge projecting posteriorly of the tips.

4. The lens of claim 3 wherein the lens is formed from a soft, foldable material.

5. An intraocular lens, comprising;
a) an optic having an anterior face;
b) a plurality of haptics having distal tips, the haptics integrally formed with the optic and projecting outwardly and posteriorly from the optic;
c) a first ridge formed the haptics between the optic and the distal tips, the first ridge projecting anteriorly of the tips, but posteriorly of the anterior face of the optic; and
d) a second ridge formed the haptics between the optic and the distal tips, the second ridge projecting posteriorly of the tips.

6. The lens of claim 5 wherein the lens is formed from a soft, foldable material.
